(19) United States
(12) Patent Application Publication
(10) Pub. No.: US 2023/0390569 A1
(43) Pub. Date: Dec. 7, 2023

(54) BONE GROWTH MODULATION USING MAGNETIC FORCES

(71) Applicant: THE REGENTS OF THE UNIVERSITY OF CALIFORNIA, Oakland, CA (US)

(72) Inventor: Vidyadhar V. Upasani, San Diego, CA (US)

(21) Appl. No.: 18/026,525

(22) PCT Filed: Aug. 27, 2021
(86) PCT No.: PCT/US2021/048011

§ 371 Date: Mar. 15, 2023

Related U.S. Application Data
(60) Provisional application No. 63/079,879, filed on Sep. 17, 2020.

(51) Int. Cl. A61N 2/00; A61B 17/68

(52) U.S. Cl. CPC A61N 2/004; A61B 17/68; A61B 2017/681; A61B 2017/00876

(57) ABSTRACT

Devices and methods that use magnetic forces to modulate bone growth are disclosed. In particular, embodiments of the presently disclosed technology apply the Heuter-Volkmann law which states that when compressive forces are exerted across a growth plate, new bone growth is inhibited, and when tensile forces are exerted across a growth plate, new bone growth is stimulated. Accordingly, embodiments use attractive forces and repulsive forces between magnets to exert compressive and tensile forces across the growth plates of a patient, thus modulating new bone growth in the patient.

### REFERENCE TO RELATED APPLICATIONS

The present application is a U.S. national phase of PCT International Patent Application No. PCT/US2021/048011, filed Aug. 27, 2021 and titled “BONE GROWTH MODULATION USING MAGNETIC FORCES”, which claims priority to U.S. Provisional Patent Application No. 63/079,879, filed Sep. 17, 2020 and titled “SPINAL IMPLANT DEVICE,” which are incorporated herein by reference in their entireties.

### TECHNICAL FIELD

The present disclosure relates generally to medical technologies, and more particularly, some embodiments relate to modulating bone growth using magnetic forces.

### DESCRIPTION OF RELATED ART

Magnets are objects that produce magnetic force fields. In particular, magnetic-field lines of force exit a magnet from a north pole and enter a south pole. Accordingly, when two magnets are placed within proximity of each other, they may exert forces upon each other depending on the orientation of their poles. Specifically, when opposite poles of the magnets are aligned, the magnets exert an attractive force on each other. By contrast, when similar poles of the magnets are aligned, the magnets exert a repelling force on each other. The magnitude of these magnetic forces may depend on such factors as orientation, size, composition of, and distance between, the magnets.

Growth plates are the areas of new bone growth in children and teens. Growth plates are typically on the ends of bones, and may add length and width to a bone as the bone grows.

### BRIEF DESCRIPTION OF THE DRAWINGS

The technology disclosed herein, in accordance with one or more various embodiments, is described with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 illustrates an example device for modulating bone growth, in accordance with various embodiments of the present disclosure.

FIG. 2 illustrates another example device for modulating bone growth, in accordance with various embodiments of the present disclosure.

FIG. 3 illustrates another example device for modulating bone growth, in accordance with various embodiments of the present disclosure.

FIG. 4 illustrates another example device for modulating bone growth, in accordance with various embodiments of the present disclosure.

FIG. 5 illustrates another example device for modulating bone growth, in accordance with various embodiments of the present disclosure.

FIG. 6 illustrates an example procedure for correcting genu valgum (i.e. “knock-knees”) using bone growth modulation, in accordance with various embodiments of the present disclosure.

FIG. 7 illustrates anterior spinal overgrowth in an example spine of a patient.

FIG. 8 illustrates example coronal plane x-rays from a posterior instrumented fusion surgery.

FIG. 9 illustrates an example anterior spinal growth tethering procedure.

FIG. 10 illustrates an example procedure for correcting idiopathic scoliosis, in accordance with various embodiments of the present application.

FIG. 11 illustrates another example procedure for correcting idiopathic scoliosis, in accordance with various embodiments of the present application.

FIG. 12 illustrates an example magnetic member embedded within an example vertebral body of a patient, in accordance with various embodiments of the present application.

FIG. 13 illustrates a device having two magnetic members coupled to an example vertebral body of a patient, in accordance with various embodiments of the present application.

The figures are not intended to be exhaustive or to limit the presently disclosed technology to the precise form disclosed. It should be understood that the presently disclosed technology can be practiced with modification and alteration, and that the disclosed technology be limited only by the claims and the equivalents thereof.

### DETAILED DESCRIPTION OF THE EMBODIMENTS

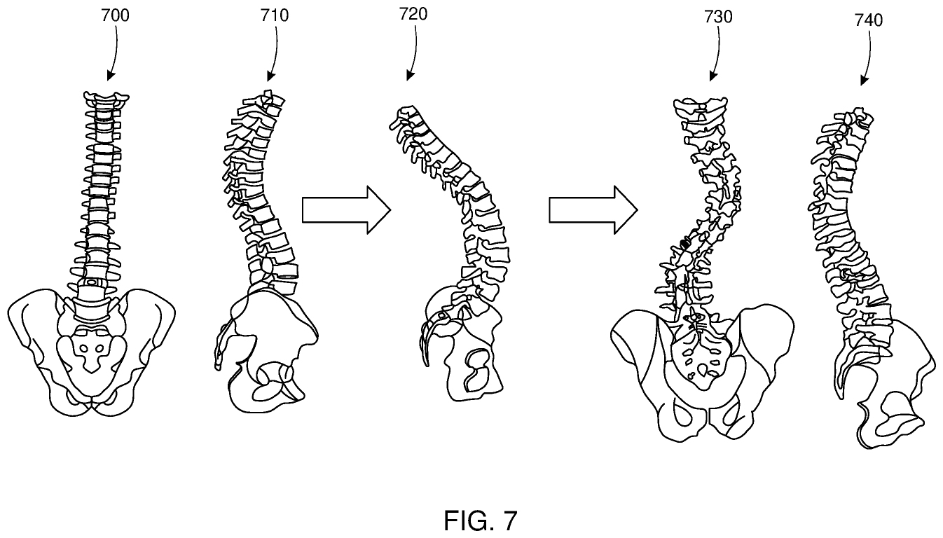

As alluded to above, growth plates are the areas of new bone growth in children and teens. Unfortunately, there are numerous disorders associated with irregular bone growth. For example, genu valgum (commonly known as “knock knee syndrome”) is a disorder involving an incorrect alignment of bones around the knee, which may be caused by asymmetric growth on the distal end of a femur. Similarly, Blount's disease, which may result in bow-leggedness, may be caused by irregular bone growth in the tibia. Another common disorder associated with irregular/asymmetric bone growth in children and teens is a spinal deformity called idiopathic scoliosis. A spine is made up of vertebral bodies. Idiopathic scoliosis is a condition where the growth plates on the front/anterior portion of the vertebral bodies may grow faster than the back/posterior portions. Also known as anterior spinal overgrowth, this condition causes a three-dimensional buckling of the spine. As can be seen in FIG. 7, the spine may develop concavities and convexities in both the coronal plane (the vertical plane which splits the body between front and back), and the sagittal plane (the vertical plane which splits the body between left and right).

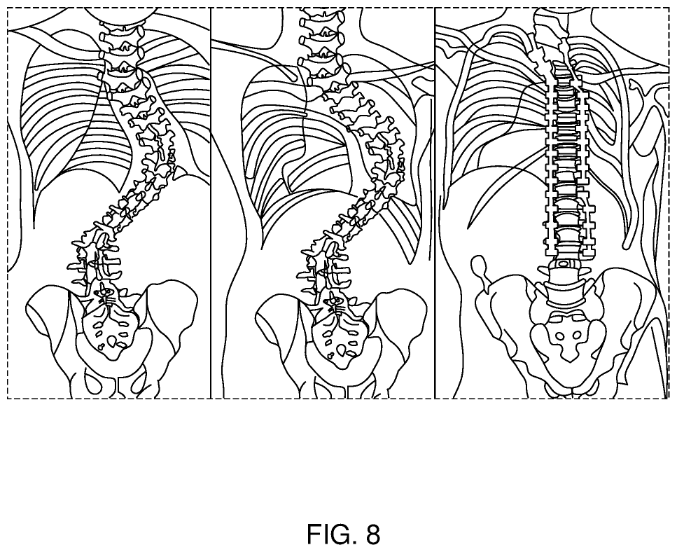

The conventional surgical procedure for correcting idiopathic scoliosis is posterior instrumented fusion. Posterior instrumented fusion is essentially a biological welding of the spine. In particular, after a spinal deformity has been corrected (or before it has progressed significantly) screws are inserted through the posterior portion of the vertebral bodies of a patient (see FIG. 8). These screws are connected by rigid metal rods (typically titanium or cobalt chrome). Then bone grafts are placed along the spine, essentially turning the spine into a continuous sheet of bone. In this way, the progression of spinal deformity caused by anterior spinal overgrowth may be stopped. However, posterior instrumented fusion is an imperfect solution for treating idiopathic scoliosis. The long term implications of turning a mobile spine into what is essentially a continuous rigid bone are still unknown. Moreover, patients who receive this treatment may face revision surgeries and/or pain and arthritis later in life.

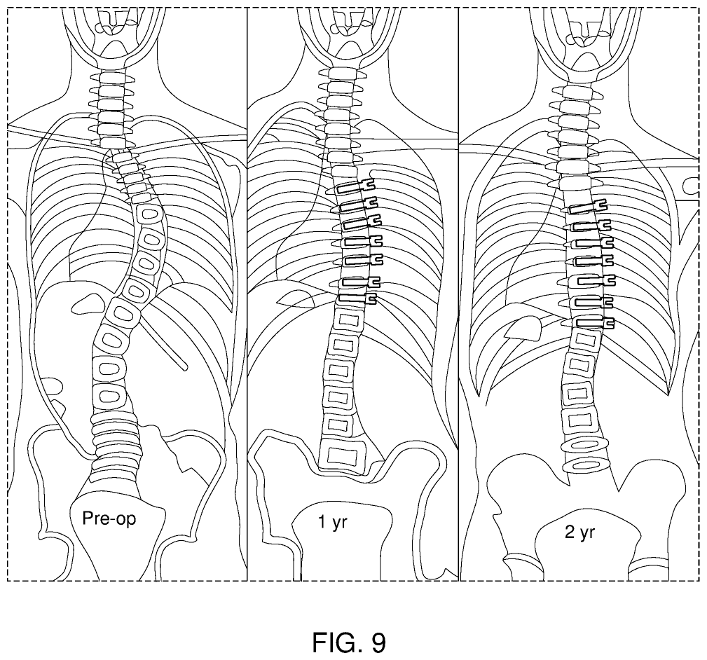

Another surgical procedure for treating idiopathic scoliosis is anterior spinal growth tethering (see FIG. 9). This surgical procedure applies the Heuter-Volkmann law, which states that when compressive forces are exerted across a growth plate, bone growth is inhibited. By contrast, when tensile forces are exerted across a growth plate, bone growth is stimulated. Accordingly, in this surgical procedure, screws are inserted into the anterior/front portion of the vertebral bodies of a patient. Tethers made of flexible materials such as polyethylene are used to connect the screws. These tethers exert compressive forces between the screws, and by extension exert compressive forces across the growth plates on the anterior portion of the vertebral bodies. In this way, anterior spinal overgrowth may be inhibited, and the spinal deformity corrected. Moreover, because flexible tethers are used instead of rigid metal rods (and because bone grafting is not involved) a patient's spine may remain mobile.

However, like posterior instrumented fusion, anterior spinal growth tethering is an imperfect treatment. In particular, the material used to tether the screws together has been observed to deteriorate in 4-6 years, which may cause a return of the spinal deformity without a corrective surgical procedure. In addition, the screws which are inserted into the anterior portion of a patient's vertebral bodies protrude into the patient's chest cavity. This can result in scarring of lung tissue, and makes corrective procedures both difficult and dangerous. Another limitation with this procedure is that it does not allow for different magnitudes of forces to be applied along different portions of the spine. In particular, it would be advantageous to apply greater compressive forces at the apex of the deformity and apply lesser compressive forces at the ends of the deformity.

Aside from the surgical procedures discussed above, other treatments for correcting irregular bone growth (and their resulting conditions such as bow-leggedness and knock knees) typically involve braces and surgeries involving the insertion of screws and plates.

Against this backdrop, embodiments of the technology disclosed herein are directed towards devices which use magnetic forces to modulate bone growth. In particular, the application of magnets can be used to apply magnetic forces to (1) exert compressive forces across a growth plate in order to inhibit bone growth; and/or (2) exert tensile forces across a growth plate in order to stimulate bone growth. These forces may be adjusted based on such factors as magnet orientation, magnet size, magnet material, and the location of magnets relative to each other. In some embodiments, the magnets may be applied to exert forces on the bones as appropriate in accordance with the Heuter-Volkmann law.

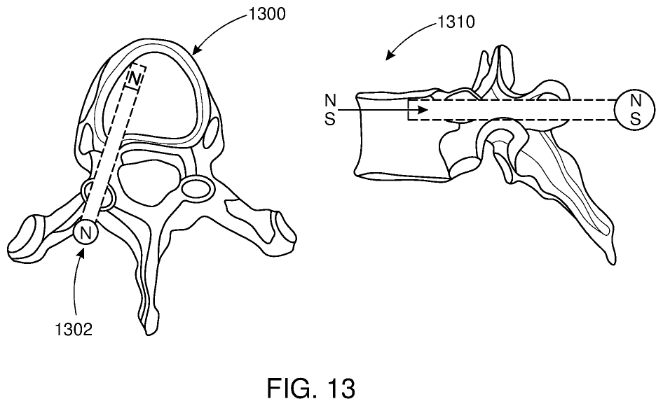

Embodiments of the presently disclosed technology may be applied to treat myriad conditions involving irregular bone growth. For example, embodiments may be used to treat knock knees, bow-leggedness or other conditions. As will be discussed in greater detail below, magnetic forces may be used to modulate vertebral bone growth in order to correct idiopathic scoliosis as well. For example, attractive forces between two magnets may be used to exert compressive forces across the growth plates on the anterior portions of the vertebral bodies of a patient. In this way, anterior spinal overgrowth may be inhibited, and the spinal deformity corrected. In another example, repulsive forces between two magnets may be used to exert tensile forces across the growth plates on the posterior portions of vertebral bodies. In this way, posterior spinal growth may be stimulated, and spinal growth imbalance may be corrected. In certain embodiments, magnetic forces may be used to exert compressive/tensile forces across both the anterior and posterior portions of a patient's vertebral bodies in order to correct spinal growth imbalances (see FIG. 13).

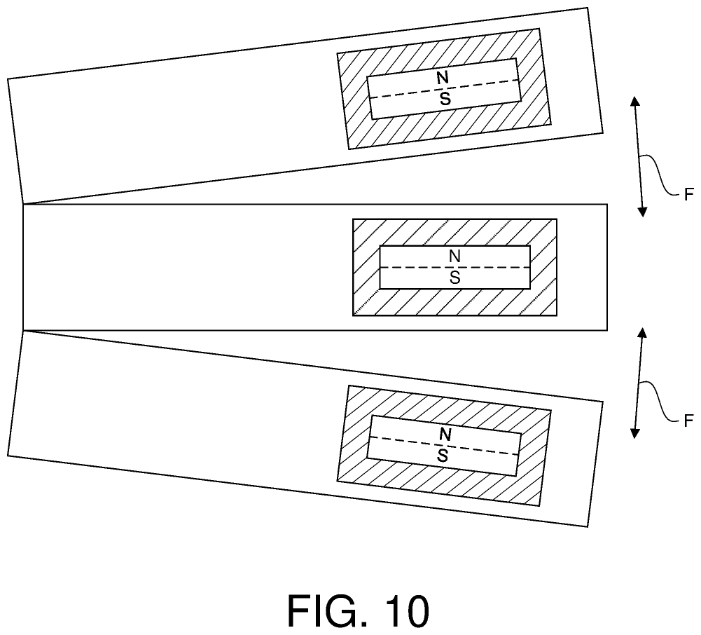

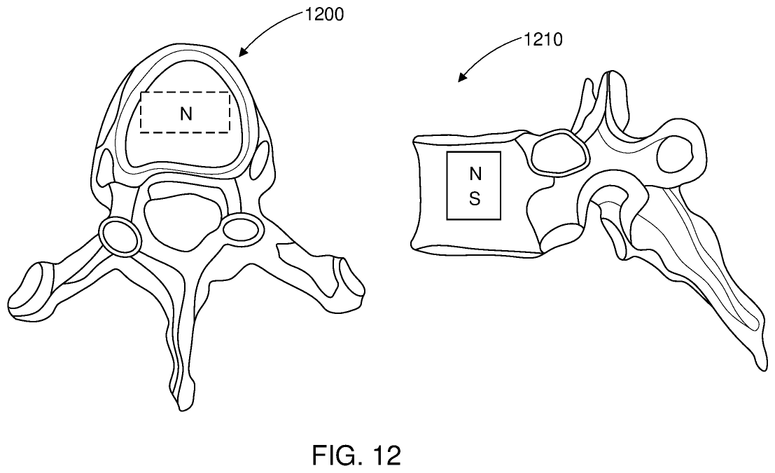

Embodiments of the presently disclosed technology have numerous advantages over conventional procedures for treating disorders associated with irregular bone growth. First, embodiments remove the mechanical connection between bones/segments required in conventional procedures. For example, embodiments may eliminate the tethers and rods used in conventional spinal correction surgeries. This allows for greater mobility for the patient, and eliminates a surgical component which may be prone to deterioration and require replacement. Eliminating this mechanical connection may also allow for a lower profile device, which in some embodiments may be embedded within a bone. For example, in the case of anterior spinal surgeries/procedures, magnetic devices may be embedded within the vertebral bodies of a patient, eliminating protrusions into the patient's chest cavity (see FIGS. 10 and 12).

Second, embodiments allow for different forces to be applied along different segments of bone(s). For example, different size/strength magnets may be used to exert greater corrective forces at the apex of a deformity, and lesser compressive forces at the end of the deformity.

Third, unlike conventional technologies, embodiments of the presently disclosed technology may be easily amplified and/or turned off. As a simple example, a magnet embedded within/attached to one bone may be rotated 90 degrees such that it does not exert a force on a magnet embedded within/attached to another bone. In other embodiments, one or more magnets may comprise electric loop circuits that have magnetic fields that can be modulated by changing the magnitude and/or direction of the electric current. Finally as alluded to above, embodiments directed towards correcting anterior spinal overgrowth may involve surgical insertions/implantations from either the anterior side of a patient, or the posterior side. This flexibility is valuable as posterior spinal surgeries are still more common, and there is a learning curve associated with anterior spinal surgeries such as anterior spinal growth tethering.

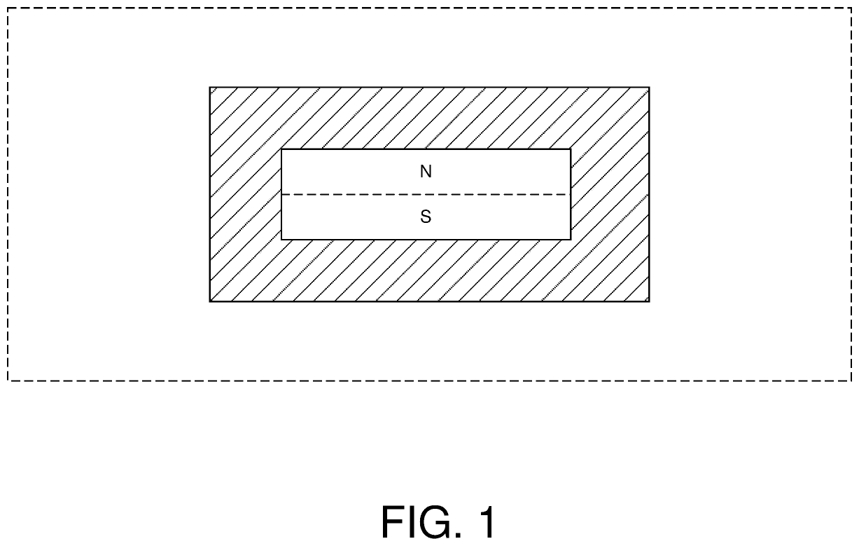

FIG. 1 illustrates a cross-sectional view of an example device for modulating bone growth, in accordance with various embodiments of the present disclosure. A magnetic member (e.g., a neodymium magnet) may be encased in a non-magnetic material, such as titanium. As is described in greater detail below, this device may be embedded within a bone of a patient.

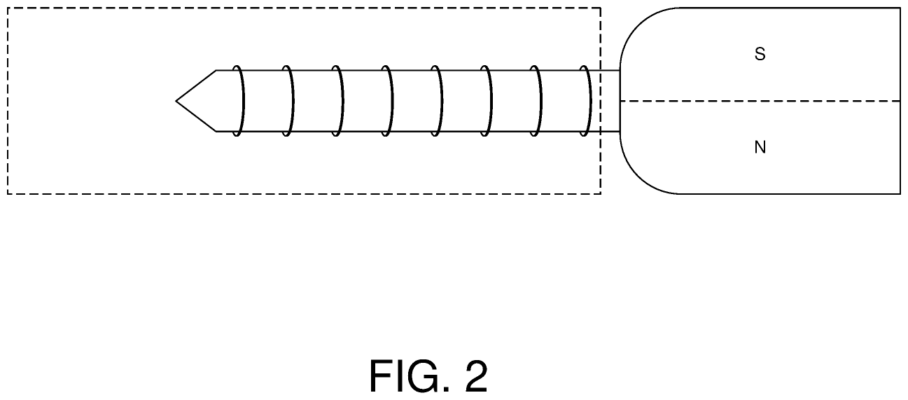

FIG. 2 illustrates another example device for modulating bone growth, in accordance with various embodiments of the present disclosure. A magnetic member may be incorporated into a screw. As illustrated, the screw may include a shank and a tulip. Accordingly, the tulip of the screw may be made from a magnetic material (e.g., neodymium magnet) with the polarities as illustrated. As is described below, the shank of the screw may be inserted into a bone in such a manner where the magnetic tulip of the screw remains outside of the bone. In embodiments, the magnetic member may be encased in a non-magnetic material such as titanium.

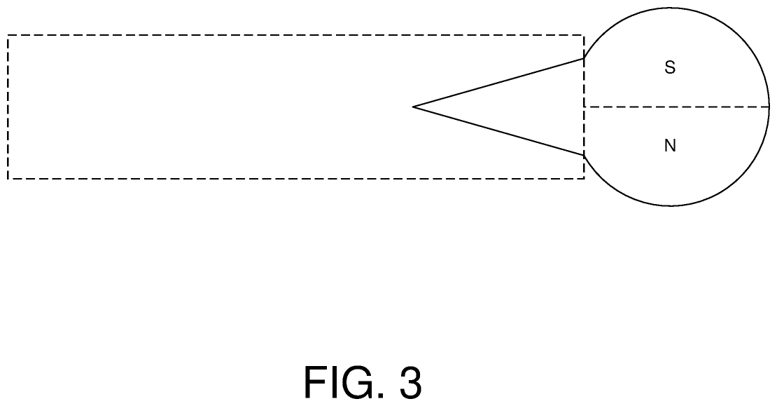

FIG. 3 illustrates another example device for modulating bone growth, in accordance with various embodiments of the present disclosure. A magnetic member may be incorporated into a head of a spike. Here, instead of a screw shank, a spike like feature may be attached to a head which is made of a suitable magnetic material, with the polarities as illustrated. As will be described below, the spike like feature may be inserted into a bone in such a manner where the magnetic head of the spike remains outside of the bone. In embodiments, the magnetic member may be encased in a non-magnetic material such as titanium.

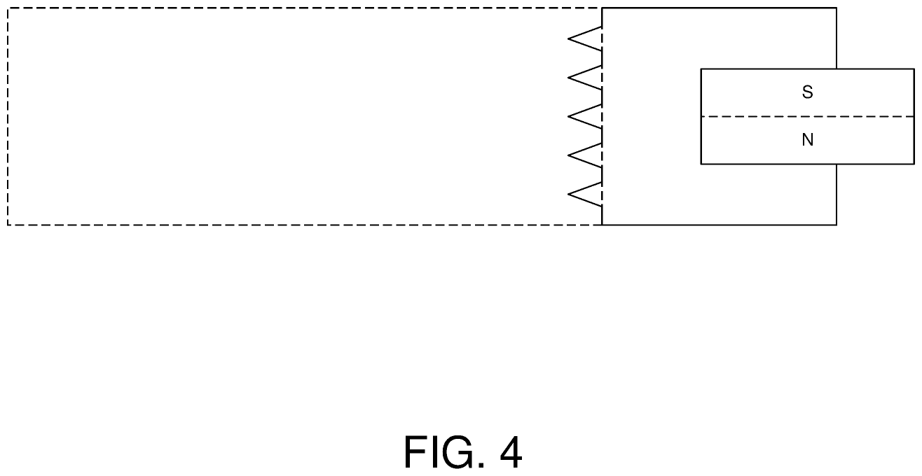

FIG. 4 illustrates another example device for modulating bone growth, in accordance with one embodiment of the present disclosure. Here, a magnetic member may be incorporated into a staple. As will be described below, the staple may be inserted into a bone in such a manner where the magnetic member remains outside of the bone. In embodiments, the magnetic member may be encased in a non-magnetic material such as titanium.

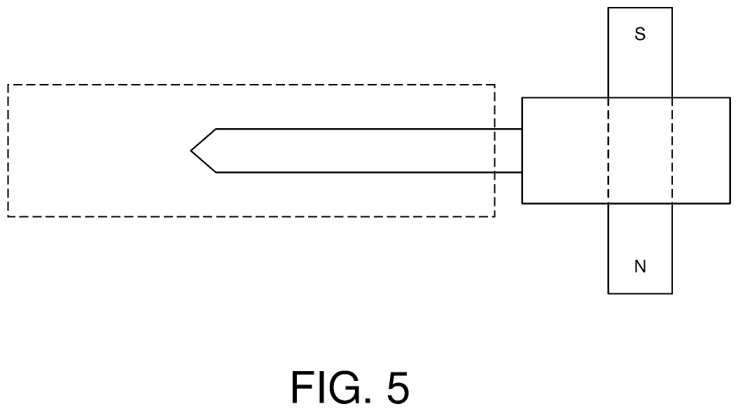

FIG. 5 illustrates another example device for modulating bone growth, in accordance with one embodiment of the present disclosure. Here, a magnetic member may exist inside of a single rod. This may enable adjacent magnetic members to be in closer proximity to generate greater forces across adjacent vertebral bodies. In embodiments, the magnetic member may be encased in a non-magnetic material such as titanium.

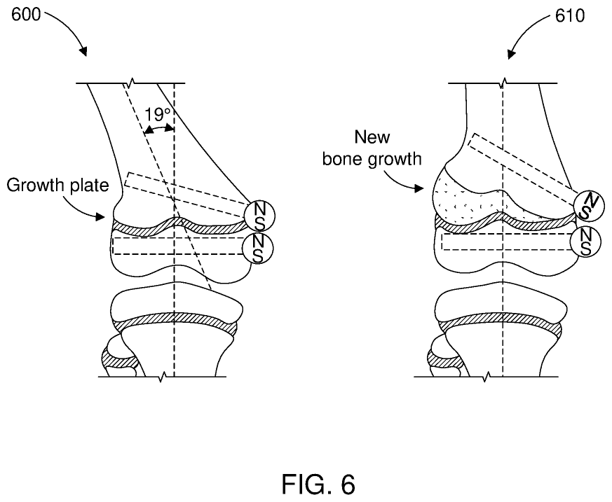

FIG. 6 illustrates an example procedure for correcting knock knees using bone growth modulation, in accordance with various embodiments of the present disclosure. Diagram 600 illustrates a femoral physis from the front/anterior view. As can be seen, the medial end (on the right) of the femur has grown faster than the lateral end (on the left). This asymmetric growth may cause the femur to deflect at an angle, such as the 19° angle depicted. In the example procedure, two magnetic devices, such as those described in conjunction with FIGS. 1-5, may be mechanically coupled to either side of the femoral physis to either inhibit bone growth on the medial end, or stimulate bone growth on the lateral end.

For example, two magnetic devices may be mechanically coupled to either side of the medial end of the femoral physis. These magnetic devices may be oriented in such a manner where opposite magnetic poles are aligned. In this way, the magnetic devices coupled to either side of the femoral physis may exert an attractive force on each other. Accordingly, compressive forces may be applied across the medial end of the femoral physis growth plate. As discussed above, these compressive forces inhibit bone growth according to the Heuter-Volkmann law. In this way, medial overgrowth may be slowed, and knock-knees may be corrected.

In another example, two magnetic devices may be mechanically coupled to either side of the lateral end of the femoral physis. These magnetic devices may be oriented in such a manner where similar magnetic poles are aligned. In this way, the magnetic devices coupled to either side of the femoral physis may exert a repulsive force on each other. Accordingly, tensile forces may be applied across the lateral end of the femoral physis growth plate. As discussed above, these tensile forces stimulate bone growth according to the Heuter-Volkmann law. In this way, lateral growth may be balanced with medial growth, and knock-knees may be corrected.

It should be understood that in either example, mechanical coupling may comprise (1) embedding the magnetic member of a magnetic device at least partially within a bone of a patient, or (2) attaching the magnetic member of a magnetic device to a bone of a patient in such a manner that the magnetic member is located outside of the bone. For example, two magnetic devices described in conjunction with FIG. 1 may be completely embedded within bone on either side of the femoral physis on the medial end. As alluded to above, these magnetic devices may be oriented in such a manner where opposite magnetic poles are aligned, thereby exerting a compressive force across the medial end of the femoral physis growth plate. In another example (and as depicted in FIG. 6), two non-magnetic screw shanks may be inserted into bone on either side of a femoral physis on the medial end. These screws may be the same/similar as those described in conjunction with FIG. 2. Accordingly, the magnetic tulips of the two screws may be located outside of the femoral physis, on the medial end. As alluded to above, these magnetic devices may be oriented in such a manner where opposite magnetic poles are aligned, thereby exerting a compressive force across the medial end of the femoral physis growth plate.

FIG. 7 illustrates anterior spinal overgrowth in an example spine of a patient. As alluded to above, anterior spinal overgrowth observed in children with idiopathic scoliosis may lead to a three-dimensional spinal deformity. Diagram 700 is a front/anterior view of the spine with minimal deformity. The spine is relatively straight in the coronal plane. Diagram 710 is a side/lateral view of the spine in a relatively normal alignment. From this view, the anterior portion of the vertebral bodies are on the right side of the spine, and the posterior portion of the vertebral bodies are on the left side. Diagram 720 is a simulated side view of the spine which illustrates the spinal imbalance that occurs with anterior spinal overgrowth.

As can be seen in the diagram, the anterior portions of the vertebral bodies (i.e. the anterior vertebral bodies) have grown faster than the posterior portions. This growth imbalance causes the anterior portion of the spine to increase in length faster than the posterior portion. As a result, pronounced concavities/convexities arise in the spine as it buckles to keep the head centered over the pelvis. Diagram 730 is a front/anterior view of the spine which illustrates the typical three-dimensional deformity present in children with idiopathic scoliosis. As can be seen, pronounced concavities/convexities result in the coronal plane.

Diagram 740 illustrates the spine of diagram 730 from the side view. As can be seen, there is a three-dimensional twisting/buckling of the spine.

FIG. 8 illustrates example coronal plane x-rays from a posterior instrumented fusion surgery. As can be seen in the figure, the deformity progresses from the initial radiograph on the left to the middle radiograph. This is then treated by screws that may be inserted into the vertebral bodies of a patient's spine. These screws may then be connected with rigid rods. In this way, the progression of spinal deformity may be stopped.

FIG. 9 illustrates an example anterior spinal growth tethering procedure. This figure illustrates an example spine from the front/anterior. As can be seen in the figure, there is pronounced concavity/convexity in the coronal plane. As illustrated, polymeric tethers are connected to screws inserted into the anterior portions of the vertebral bodies of the spine. These tethers may apply compressive forces across the growth plates of the anterior portions of the vertebral bodies, inhibiting bone growth. However, when vertebral body tethering is used for anterior overgrowth correction, the tethers (typically made of polymers like polyethylene) may be prone to fraying and breakage which leads to revision surgeries. On the other hand, if a rod or metal cord is used, it can result in too much compression on the anterior vertebral segments, resulting in vertebral body fusion or disc degeneration.

Additionally, as alluded to above, idiopathic scoliosis correction by this method cannot vary the magnitude of the compressive force applied to a particular vertebral segment. Accordingly, embodiments of the presently disclosed technology improve upon this existing technology by allowing for greater magnetic/compressive forces to be applied at the apex of the deformity, and lesser magnetic/compressive forces to be applied at the end of the deformity.

FIG. 10 illustrates an example procedure for correcting idiopathic scoliosis (i.e. anterior spinal overgrowth) in accordance with various embodiments of the present application. The figure illustrates an effected spine from the front/anterior view. As can be seen, there is a convexity to the spine in the coronal plane (on the right side of the figure). In the example procedure, magnetic devices, such as those described in conjunction with FIG. 1, may be embedded within the anterior vertebral bodies of the spine. The magnetic devices may be oriented such that opposite magnetic poles are aligned. In this way, the magnetic devices embedded within adjacent anterior vertebral bodies may exert attractive forces on each other. Accordingly, compressive forces may be applied across the growth plates of the anterior vertebral bodies. As discussed above, these compressive forces may be applied to inhibit bone growth according to the Heuter-Volkmann law. In this way, anterior spinal overgrowth may be slowed, and idiopathic scoliosis may be corrected.

As depicted in the example procedure, the magnetic devices are embedded on the side of the anterior vertebral bodies closer to the convex deformity in the coronal plane. This may be a preferred location for the embedded magnetic devices in some embodiments. However, in other embodiments the magnetic devices may be embedded in other locations within the anterior vertebral body.

As alluded to above, in some embodiments, greater compressive forces may be applied along different segments of the spine. For example, greater compressive forces may be exerted on the growth plates of the anterior vertebral bodies near the apex of the deformity. This may be achieved in numerous ways. For example, the size, strength, number (or a combination of the size, strength and number) of the magnets embedded in anterior vertebral bodies near the apex of the deformity may be greater than the size/strength/number of the magnets embedded in anterior vertebral bodies farther removed from the apex of the deformity. Similarly, orientation of the magnets may also be adjusted such that the attractive forces between magnets embedded near the apex of the deformity are greater than the attractive forces between magnets embedded farther away from the apex of the deformity.

Additionally, it should be understood that embodiments of the presently disclosed technology may be used to modulate growth of posterior vertebral bodies as well. For example, magnetic devices in accordance with embodiments of the presently disclosed technology may be embedded within the posterior vertebral bodies of an effected spine. Here, the magnetic devices may be oriented such that like magnetic poles align. In this way, the magnetic devices embedded within adjacent posterior vertebral bodies may exert repulsive forces on each other. Accordingly, tensile forces may be applied across the growth plates of the posterior vertebral bodies, stimulating bone growth in the posterior vertebral bodies. In this way, spinal growth may be balanced, and idiopathic scoliosis may be corrected.

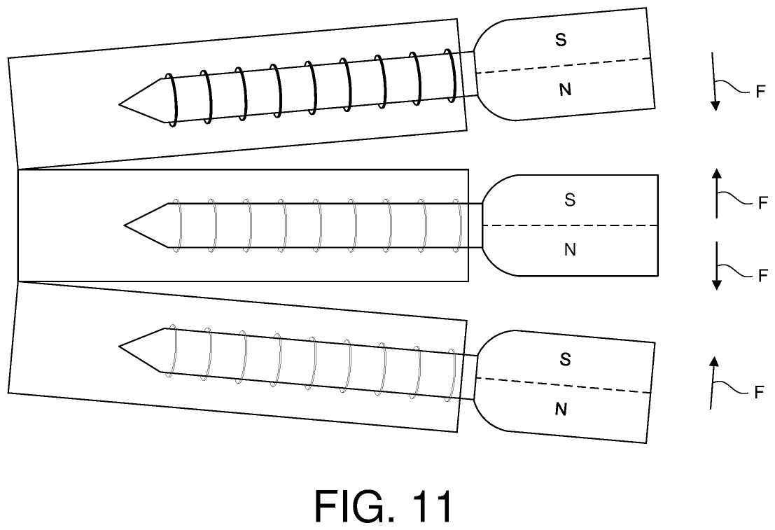

FIG. 11 illustrates another example procedure for correcting anterior spinal overgrowth in accordance with various embodiments of the present application. Like FIG. 10, this figure illustrates an effected spine from the front/anterior view. As can be seen, there is a convexity to the spine in the coronal plane. In the example figure, magnetic devices such as those described in conjunction with FIG. 2 may be attached to anterior vertebral bodies in such a manner that the magnetic members remain outside the bone. It should be understood that any of the devices described in conjunction with FIGS. 3-5 may be attached in the same/similar manner. Here, the attached magnetic devices may be oriented in such a manner where opposite magnetic poles are aligned. In this way, the magnetic devices attached to adjacent anterior vertebral bodies may exert attractive forces on each other. Accordingly, compressive forces may be applied across the growth plates of the anterior vertebral bodies. As discussed above, the magnetic devices may be positioned such that these compressive forces inhibit bone growth according to the Heuter-Volkmann law. In this way, anterior spinal overgrowth may be slowed/corrected.

As depicted in the example procedure, the magnetic devices are located on the side of the anterior vertebral bodies closer to the convex deformity in the coronal plane. This may be a preferred location for the attached magnetic devices in some embodiments. However, in other embodiments the magnetic devices may be attached to other locations on the anterior vertebral body.

As described in conjunction with FIG. 10, this same/similar procedure may be applied to the posterior vertebral bodies of an effected spine. As alluded to above, under a posterior approach, the magnetic devices may be oriented such that like poles are aligned. In this way, tensile forces may be exerted across the growth plates of the posterior vertebral bodies, balancing spinal growth. Likewise, greater compressive/tensile forces may be applied along different segments of the spine in the same/similar manner to that described in conjunction with FIG. 10. In this way, greater compressive/tensile forces may be applied to vertebral bodies closer to the spinal deformity.

FIG. 12 illustrates an example magnetic member embedded within the anterior portion of an example vertebral body, in accordance with various embodiments of the present application. Diagram 1200 is a top view of the vertebral body, and diagram 1210 is a side view of the vertebral body, where the anterior portion is located on the left.

FIG. 13 illustrates a device having two magnetic members coupled to an example vertebral body of a patient, in accordance with various embodiments of the present application. Diagram 1300 is a top view of the vertebral body, and diagram 1310 is a side view of the vertebral body, where the posterior portion is located on the right. As illustrated, a single device may be used to couple a magnetic member to each of the anterior portion and the posterior portion of the same vertebral body. For example, a screw may have a segment of a shank which is magnetic, and a segment of the shank which is non-magnetic. As illustrated in FIG. 13, the magnetic segment of the screw shank may be located at the distal end. The screw may also have a magnetic tulip. Such a screw may be inserted through the pedicle of the vertebral body from the posterior side. The magnetic tulip may be located outside of the vertebral body, on the posterior side, and the magnetic segment of the shank may be embedded within the anterior portion of the vertebral body. Accordingly, similar screws may be inserted into adjacent vertebral bodies of a patient in the same/similar manner. The magnetic segments of each screw shank may be oriented in a manner where opposite poles of adjacent magnets are aligned. In this way, the magnetic shank segments may exert attractive forces upon each other, thereby exerting compressive forces across the growth plates of the anterior vertebral bodies. By contrast, the magnetic tulips of each screw may be oriented in a manner where similar poles of adjacent magnets are aligned. In this way, the magnetic tulips may exert repulsive forces upon each other, thereby exerting tensile forces across the growth plates of the posterior vertebral bodies.

As used herein, the term component may describe a given unit of functionality that may be performed in accordance with one or more embodiments of the present application. In embodiments, a component may be implemented utilizing any form of hardware. In implementation, the various components described herein may be implemented as discrete components or the functions and features described may be shared in part or in total among one or more components. In other words, as would be apparent to one of ordinary skill in the art after reading this description, the various features and functionality described herein may be implemented in any given application and may be implemented in one or more separate or shared components in various combinations and permutations. Even though various features or elements of functionality may be individually described or claimed as separate components, one of ordinary skill in the art will understand upon studying the present disclosure that these features and functionality may be shared among one or more hardware elements, and such description shall not require or imply that separate hardware components are used to implement such features or functionality.

While various embodiments of the disclosed technology have been described above, it should be understood that they have been presented by way of example only, and not of limitation. Likewise, the various diagrams may depict an example architectural or other configuration for the disclosed technology, which is done to aid in understanding the features and functionality that can be included in the disclosed technology. The disclosed technology is not restricted to the illustrated example architectures or configurations, but the desired features can be implemented using a variety of alternative architectures and configurations. Indeed, it will be apparent to one of skill in the art how alternative functional, physical partitioning and configurations can be implemented to implement the desired features of the technology disclosed herein. Also, a multitude of different constituent component names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosed technology is described above in terms of various exemplary embodiments and implementations, it should be understood that the various features, aspects and functionality described in one or more of the individual embodiments are not limited in their applicability to the embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “component” does not imply that the components or functionality described or claimed as part of the component are all configured in a common package. Indeed, any or all of the various components of a component can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration

What is claimed is:

1. A device comprising:
a first magnetic member configured to be mechanically coupled to a first bone in a patient, the first bone having a first growth plate; and
a second magnetic member configured to be implanted in the patient, wherein placement of the first and second magnetic members is chosen to modulate bone growth by exerting at least one of a compressive force and a tensile force across the first growth plate when mechanically coupled to the first bone and implanted in the patient respectively.

2. The device of claim 1, wherein:
the second magnetic member is configured to be mechanically coupled to a second bone in the patient, the second bone having a second growth plate; and
placement of the first and second magnetic members is chosen to modulate bone growth by exerting at least one of a compressive force and a tensile force across the second growth plate when mechanically coupled to the first bone and the second bone respectively.

3. The device of claim 1, wherein the second magnetic member is configured to be mechanically coupled to the first bone.

4. The device of claim 1, wherein the mechanical coupling comprises being at least partially embedded within the first bone.

5. The device of claim 1, wherein the mechanical coupling comprises being mechanically attached to, but located outside of the first bone.

6. The device of claim 4, wherein the device further comprises a non-magnetic enclosure that encases the first magnetic member.

7. The device of claim 6, wherein the non-magnetic enclosure is made of titanium.

8. The device of claim 5, wherein:
the first magnetic member is a tulip of a screw, the screw having the magnetic tulip and a non-magnetic shank; and
the non-magnetic shank is configured to be inserted into the first bone.

9. A device comprising:
a first magnetic member configured to be embedded within an anterior portion of a first vertebral body of a patient, the anterior portion of the first vertebral body having a top and bottom growth plate; and
a second magnetic member configured to be embedded within an anterior portion of a second vertebral body of the patient, the anterior portion of the second vertebral body having a top and bottom growth plate, wherein placement of the first and second magnetic members is chosen to modulate bone growth by exerting a compressive force across the bottom growth plate of the first vertebral body and the top growth plate of the second vertebral body when embedded within the anterior portions of the first and second vertebral bodies respectively.

10. The device of claim 9, wherein the first and second vertebral bodies are adjacent to each other on a spine of the patient.

11. The device of claim 10, further comprising a third magnetic member configured to be embedded within an anterior portion of a third vertebral body of the patient, the anterior portion of the third vertebral body having a top and bottom growth plate, wherein:
the second and third vertebral bodies are adjacent to each other on the spine of the patient; and
placement of the second and third magnetic members is chosen to modulate bone growth by exerting a compressive force across the bottom growth plate of the second vertebral body and the top growth plate of third vertebral body when embedded within the anterior portions of the second and third vertebral bodies respectively.

12. The device of claim 11, wherein the compressive force exerted by the first and second magnetic members on the bottom growth plate of the first vertebral body and the top growth plate of the second vertebral body is greater in magnitude than the compressive force exerted by the second and third magnetic members on the bottom growth plate of the second vertebral body and the top growth plate of the third vertebral body.

13. The device of claim 9, wherein:
the first magnetic member is encased in a first non-magnetic enclosure; and
the second magnetic member is encased in a second non-magnetic enclosure.

14. A method comprising:
mechanically coupling a first magnetic member to a first bone in a patient, the first bone having a first growth plate; and
implanting a second magnetic member in the patient, wherein placement of the first and second magnetic members is chosen to modulate bone growth by exerting at least one of a compressive force and a tensile force across the first growth plate when coupled to the first bone and implanted within the patient respectively.

15. The method of claim 14, wherein:
implanting the second magnetic member in the patient comprises mechanically coupling the second magnetic member to a second bone, the second bone having a second growth plate; and
placement of the first and second magnetic members is chosen to modulate bone growth by exerting at least one of a compressive force and a tensile force across the second growth plate when coupled to the first bone and the second bone respectively.

16. The method of claim 14, wherein mechanically coupling the first magnetic member to the first bone comprises embedding the first magnetic member at least partially within the first bone.

17. The method of claim 16, wherein the first magnetic member is encased in a non-magnetic enclosure.

18. The method of claim 17, wherein the non-magnetic enclosure is made of titanium.

19. The method of claim 14, wherein mechanically coupling the first magnetic member to the first bone comprises mechanically attaching the first magnetic member to the first bone in a manner where the first magnetic member is located outside the first bone.

20. The method of claim 19, wherein:
the first magnetic member is a tulip of a screw, the screw having the magnetic tulip and a non-magnetic shank; and
